(19) United States
(12) Patent Application Publication
(10) Pub. No.: US 2017/0049149 A1
(43) Pub. Date: Feb. 23, 2017

(54) Solar-powered multi-purpose vaporizer

(71) Applicant: Jessica Carty, Woodbridge, VA (US)

(72) Inventor: Jessica Carty, Woodbridge, VA (US)

(21) Appl. No.: 14/821,132

(22) Filed: Aug. 7, 2015

Related U.S. Application Data
(60) Provisional application No. 62/027,039, filed on Jul. 21, 2014.

(51) Int. Cl. A24F 47/00; A61M 15/00; A61M 15/06; A61M 11/04

(52) U.S. Cl. CPC A24F 47/008; A61M 11/042; A61M 15/0021; A61M 15/06; A61M 2205/8293

(57) ABSTRACT

A solar-powered multi-purpose vaporizer contains chambers for smoking herbs, waxes and oils. The vaporizer is shaped like a ballpoint pen, with a solar panel on the top surface which may be used to recharge the battery, two internal chambers are provided. The outer chamber may be used for smoking herbs, waxes, and oils, and provides a plastic screen to prevent messes. The inner chamber may be used to store about one half gram of dried smoking herbs, waxes, or oils ready for use.

### CROSS-REFERENCE TO RELATED APPLICATIONS

This Application claims the benefit of U.S. Provisional Application No. 62/027,039, filed Jul. 21, 2014, which is hereby incorporated by reference.

### STATEMENT REGARDING FEDERALLY SPONSORED RESEARCH OR DEVELOPMENT

Not Applicable

### PARTIES TO A JOINT RESEARCH AGREEMENT

Not Applicable

### REFERENCE TO SEQUENCE LISTING, A TABLE, OR A COMPUTER PROGRAM LISTING COMPACT DISK APPENDIX

Not Applicable

### BACKGROUND OF THE INVENTION

The invention relates generally to smoking accessories, and in particular to a solar-powered multi-purpose vaporizer. The electric vaporizer, or e-cigarette, has significantly reduced the risks of second-hand smoke for millions of Americans. As the use of e-liquid increases as a delivery vehicle for the use of nicotine, the use of other smoking oils, herbs, and waxes is also increasing. The medicinal use of marijuana has been legalized in 23 states, including New York and California, and its recreational use is legal in two states. For people using more than one type of these substances, the many different smoking implements can take up space and be difficult to keep sorted. Also, keeping them supplied with batteries can become a chore. A solar-powered multi-purpose vaporizer, capable of operating on solar power alone and smoking many types of smoking oils, herbs, and waxes, would resolve these problems.

### SUMMARY OF THE INVENTION

Accordingly, the invention is directed to a solar-powered multi-purpose vaporizer. The vaporizer is shaped like a ballpoint pen, with a solar panel on the top surface which may be used to recharge the battery. Two internal chambers are provided. The outer chamber may be used for smoking herbs, waxes, and oils, and provides a plastic screen to prevent messes. The inner chamber may be used to store one half gram of dried smoking herbs, waxes, or oils ready for use.

Additional features and advantages of the invention will be set forth in the description which follows, and will be apparent from the description, or may be learned by practice of the invention. The foregoing general description and the following detailed description are exemplary and explanatory and are intended to provide further explanation of the invention.

### BRIEF DESCRIPTION OF THE DRAWINGS

The accompanying drawings are included to provide a further understanding of the invention and are incorporated into and constitutes a part of the specification. They illustrate one embodiment of the invention and, together with the description, serve to explain the principles of the invention.

FIG. 1 is a top view of the first exemplary embodiment, displaying the vaporizer 10, the solar panel 11, the outer chamber 12, the inner chamber 13, and the mouthpiece 14.

FIG. 2 is a bottom view of the first exemplary embodiment, displaying the vaporizer 10, the outer chamber 12, the inner chamber 13, and the mouthpiece 14.

FIG. 3 is a bottom view of the first exemplary embodiment in a disassembled state, displaying the vaporizer 10, the outer chamber 12, the screen 12A, the cap 12B, the inner chamber 13, and the mouthpiece 14.

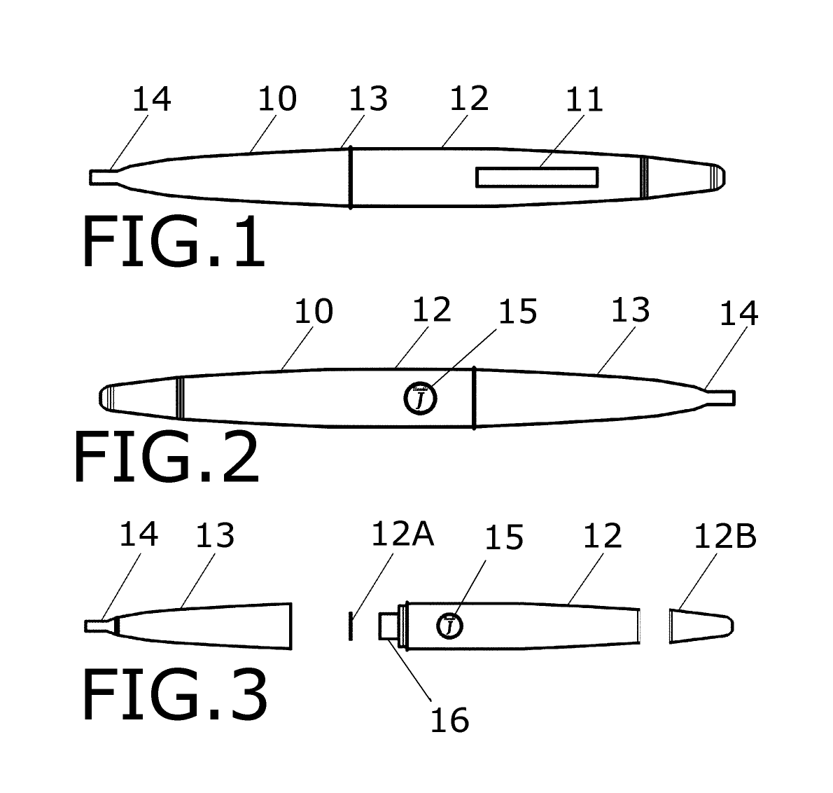

### DETAILED DESCRIPTION OF THE INVENTION

Referring now to the invention in more detail, the invention is directed to a solar-powered multi-purpose vaporizer 10.

The first exemplary embodiment is comprised of a vaporizer 10 which is preferably shaped like a ballpoint pen, and is preferably 4.5 inches in length. The vaporizer 10 provides a solar panel 11 on the top surface which may be used to recharge the battery. Two chambers are provided. The outer chamber 12 may be used for smoking herbs, waxes, and oils, and provides a plastic screen 12A to separate the smoking herbs, waxes, and oils from the atomizer 16. The inner chamber 13 may be used to store about one half gram of dried smoking herbs, ready for use. The atomizer 16 is located between the inner chamber 12 and outer chamber 13. The inner chamber may optionally be separated from the outer chamber 12 and atomizer 16 by a buffer piece; the buffer piece would provide an additional thermal barrier between the stored material in the inner chamber 13 and the heated materials in the outer chamber 12A.

A cap 12B on the outer chamber 12 contains the battery. The atomizer is located at the center of the vaporizer 10, near the screen 12A. Optionally, the atomizer may provide an automatic shutoff mechanism which deactivates the atomizer after a pre-determined period of continuous use, such as six minutes. Due to its double chambered construction, the vaporizer 10 is virtually odorless and the outer surface remains cool to the touch.

To use the first exemplary embodiment, the user may store smoking herbs in the outer chamber 12 and transfer them to the inner chamber 13 for smoking. The user presses the activator button 15 to activate the atomizer 16. The heat from the atomizer 16 vaporizes the smoking herbs in the inner chamber 13. The user then may inhale the vapor through the mouthpiece 14. Alternately, the user may smoke oils or waxes, using the screen 12A to separate the oil or wax within the inner chamber 13 from the atomizer 16.

The vaporizer 10 is preferably manufactured from rigid, durable materials which are heat and corrosion resistant, such as stainless steel, aluminum alloy, brass, and copper alloy. Components and methods listed above are preferable, but artisans will recognize that alternate components and methods could be selected without altering the scope of the invention.

While the foregoing written description of the invention enables one of ordinary skill to make and use what is presently considered to be the best mode thereof, those of ordinary skill in the art will understand and appreciate the existence of variations, combinations, and equivalents of the specific embodiment, method, and examples herein. The invention should, therefore, not be limited by the above described embodiment, method, and examples, but by all embodiments and methods within the scope and spirit of the invention.

I claim:

1. A solar-powered multi-purpose vaporizer, comprising:
(a) a solar panel;
(b) a rechargeable battery;
(c) an outer chamber;
(d) an inner chamber;
(e) a mouthpiece connected to said inner chamber;
(f) an activator button;
(g) an atomizer located between said outer chamber and said inner chamber;
(f) said solar panel, rechargeable battery, activator button, and atomizer being in electrical communication via a wired connection;
(g) said solar panel being configured to charge said rechargeable battery when exposed to sunlight;
(h) said atomizer being configured to heat said inner chamber when said activator button is pressed;

2. The solar-powered multi-purpose vaporizer of claim 1, wherein the solar-powered multi-purpose vaporizer is shaped like a ballpoint pen.

3. The solar-powered multi-purpose vaporizer of claim 2, wherein the solar-powered multi-purpose vaporizer is four and a half inches in length.

4. The solar-powered multi-purpose vaporizer of claim 1, further comprising a screen;
said screen being configured to separate said atomizer and said inner chamber.

5. The solar-powered multi-purpose vaporizer of claim 1, further comprising a buffer piece; said buffer piece being located between said atomizer and said outer chamber and configured to insulate said outer chamber from said atomizer; said inner chamber being removably affixed to said buffer piece; and said outer chamber being removably affixed to said buffer piece.

6. The solar-powered multi-purpose vaporizer of claim 1, wherein said mouthpiece is attached to an end of said inner chamber; said atomizer is permanently attached to said outer chamber on an atomizer end; said outer chamber is removably attached via the atomizer end to another end of said inner chamber opposite said mouthpiece; said battery is located in a cap; said cap being removably affixed to another end of said outer chamber opposite said atomizer end.

7. The solar-powered multi-purpose vaporizer of claim 1, wherein said atomizer is configured to automatically deactivate after a pre-determined period of time.

8. The solar-powered multi-purpose vaporizer of claim 5, wherein said atomizer is configured to automatically deactivate after a pre-determined period of time.

9. The solar-powered multi-purpose vaporizer of claim 6, wherein said atomizer is configured to automatically deactivate after a pre-determined period of time.

10. The solar-powered multi-purpose vaporizer of claim 1, wherein said outer chamber in insulated against heat.

11. The solar-powered multi-purpose vaporizer of claim 1, wherein said mouthpiece provides an opening into said inner chamber.

12. The solar-powered multi-purpose vaporizer of claim 3, wherein said mouthpiece provides an opening into said inner chamber.

13. The solar-powered multi-purpose vaporizer of claim 5, wherein said mouthpiece provides an opening into said inner chamber.

14. The solar-powered multi-purpose vaporizer of claim 6, wherein said mouthpiece provides an opening into said inner chamber.
